(19) United States
(12) Patent Application Publication
(10) Pub. No.: US 2012/0283304 A1
(43) Pub. Date: Nov. 8, 2012

(54) Formulations of Temozolomide for Parenteral Administration

(75) Inventors: Manik Reddy Pullagurla, Hyderabad (IN); Jagadeesh Babu Rangisetty, Hyderabad (IN)

(73) Assignee: SAHAJ LIFE SCIENCES PVT. LTD., Hyderabad (IN)

(21) Appl. No.: 13/518,418

(22) PCT Filed: Dec. 22, 2010
(86) PCT No.: PCT/IN2010/000845

§ 371 Date: Jul. 16, 2012

(30) Foreign Application Priority Data
Dec. 23, 2009 (IN) 3160/CHE/2009

(51) Int. Cl. A61K 31/4188; A61P 35/00

(52) U.S. Cl. 514/393

(57) ABSTRACT

The present invention relates to a parenteral formulation of Temozolomide comprising an excipient selected from the group consisting of bulking agents, buffers, pH adjusting agents, with the proviso that the formulation is free of dissolution enhancing agents.

The present invention relates to novel formulations of Temozolomide for parenteral administration.

### BACKGROUND OF THE INVENTION

Temozolomide is chemically known as 3-methyl-8-carbamoyl-imidazo[5,1-d]-1,2,3,5-tetrazin-4(3H)-one and has the following structure:

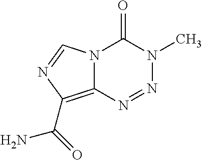

The methods of preparation of Temozolomide are described, for example, in U.S. Pat. No. 5,260,291; The Merck Index on CD-ROM, Version 12:3, 1999; Merck & Co. Inc., Whitehouse Station, N.J., USA. Published on CD-ROM by Chapman and Hall/CRC; Stevens et al. J. Med. Chem. 1984, 27, 196-201; Baig and Stevens J. Chem. Soc. Perkin Trans. 11987, 675-670; J. Chem. Soc., Chem. Commun. 1994, 1687-1688; Clark et al. J. Med. Chem. 1995, 38, 1493-1504; Newlands et al. Cancer Treatment Reviews 1997 23, 35-61; Brown et al. J. Med. Chem. 2002, 45, 5448-5457.

Temozolomide is a prodrug and is rapidly hydrolysed into 5-(3-methyltriazen-1-yl imidazole-4-carboxamide (MTIC). The compound is known for its anti-tumor effects. Temozolomide is approved for treating newly diagnosed glioblastoma multiforme concomitantly with radiotherapy and then as maintenance treatment and also for treating refractory anaplastic astrocytoma in patients with unsuccessful treatment with nitrosourea and procarbazine.

Temozolomide is only slightly soluble in water. The compound is stable at acidic pH less than 5 but is unstable at pH greater than 7. Temozolomide was originally approved for use as capsules intended for oral administration. Recently it was also approved for intravenous administration and is made available as a lyophilised formulation. This approved formulation contains dissolution enhancing additives to enhance solubility of drug substance in formulation.

U.S. Pat. No. 6,251,886 discloses microcrystalline suspension compositions of Temozolomide that can be administered by any number of means, including, e.g., intrathecally, intraventricularly, intraperitoneally, intrapleurally, intravenously, or by administration into an artery that supplies blood to a region of the body. These formulations are not preferable for intravenous administration because any change in the particle size in suspension can cause adverse effects and severe irritation to the patient.

U.S. Pat. No. 6,987,108 discloses a Temozolomide formulation comprising the active, at least one aqueous diluent, and at least one dissolution enhancing agent sufficient to substantially dissolve said Temozolomide. The patent specifically discloses lyophilised formulations of Temozolomide which are to be reconstituted with a specific diluent solution before administration. The formulations disclosed in this patent have the inherent disadvantage of requiring many excipients and more particularly excipients like polysorbate which are known to cause adverse effects and irritation at the site of injection.

The product approved for use in US contains mannitol, L-threonine, polysorbate 80, sodium citrate dihydrate and hydrochloric acid as inactive ingredients. The lyophilised product needs to be stored at 2-8° C. throughout the shelf life.

Hence there is an immediate need for improved formulations of Temozolomide for intravenous administration that are easier to manufacture and require less number of excipients and are stable when stored below 25° C.

### OBJECTIVE OF THE INVENTION

Another objective of the invention is to provide a formulation of Temozolomide injection that does not need a dissolution enhancing agent.

Another objective of the invention is to provide compositions of Temozolomide injection that have a pH of 2 to 6 and have Osmolality between 150 to 800 mOsm/Kg.

Another objective of the invention is to provide a formulation of Temozolomide for intravenous administration that is stable below 25° C.

Yet another objective of the invention is to provide suitable primary packaging configuration for the product.

Yet another objective of the invention is to provide ready to use solution formulations of Temozolomide injection which do not require reconstitution before administration.

### DETAILED DESCRIPTION OF THE INVENTION

The pharmaceutical formulation of the present invention comprises Temozolomide for intravenous administration in the dose ranging from 5 mg to 1000 mg/vial. More preferably the formulation comprises Temozolomide in a dose ranging from 80 mg to 800 mg/vial and more preferably 100 mg/vial.

The formulations of this invention do not need any dissolution enhancing aids taught in the prior art as a requirement to enhance dissolution of Temozolomide. The formulations further have such physico chemical attributes that will minimise the inconvenience during usage. For instance, the formulations have a pH between 2 and 6 and have Osmolality between 150 to 800 mOsm/Kg. The formulations can further be stored below 25° C.

The inventors have found that the pharmaceutical formulation of the present invention can be made by any of the following processes like lyophilisation, vacuum drying or spray drying or dry powder filling.

Alternately the formulation can be designed as a ready to use solution for intravenous administration.

### Lyophilised Compositions

Lyophilization, also known as freeze-drying, is a process whereby water is sublimed from a composition after it is frozen. In this process, pharmaceutical and biological agents that are relatively unstable in an aqueous solution over a period of time can be placed into dosage containers in an easily processed liquid state, dried without the use of damaging heat and stored in a dried state for extended periods. The pharmaceutical formulation of the present invention can be made by lyophilising Temozolomide with suitable excipients.

The lyophilisation process for making Temozolomide injection involves the following steps:
(a) Dissolve excipients and buffer in water for injection
(b) Temozolomide is dissolved in above solution in a concentration of between about 0.1% w/v and about 5% w/v
(c) The Temozolomide preparation from (b) is sterile filtered into a previously sterilized container
(d) The Temozolomide preparation from (c) is rapidly lowered to a temperature below at least −15° C.
(e) The temperature of the Temozolomide preparation from (d) is raised to between <0° C. and about 40° C.
(f) The Temozolomide preparation from (e) is subjected to an environment in which the pressure under vacuum of <400 millitorr.
(g) The temperature of the environment ranges from −45° C. and +50° C., subliming the water from the preparation resulting in the recovery of the product having a moisture content of not more than 6.0 percent.

Temozolomide formulation of the present invention may contain pharmaceutically acceptable bulking agents for lyophilisation. The suitable bulking agents which can be included in the formulation are mannitol, lactose, sucrose, sodium chloride, trehalose, dextrose, starch, hydroxyethylstarch (hetastarch), cellulose, cyclodextrins, glycine, or mixtures thereof.

In a preferred embodiment, the bulking agent in the pharmaceutical formulation is mannitol or sodium chloride or sorbitol or sucrose or dextrose or lactose. When a bulking agent is used in the pharmaceutical formulation, the proportion can range from about 5 wt % to about 90 wt %, preferably from about 15 wt % to about 85 wt %.

In another embodiment, the pharmaceutical formulation further comprises at least one buffer.

Suitable buffers which can be included in the pharmaceutical formulation include citrate buffers, acetate buffers, phosphate buffers, Acetic acid, Lactic acid, amino acids, tris-buffer, meglumine and the like, and pH adjusting agents that are acidic or alkaline in nature.

The formulation may additionally involve a solvent system for making the solution for lyophilisation. This system can be water or a mixture of water and an organic solvent like ethanol or tertiary-butyl alcohol.

### Vacuum Dried Compositions

The formulations for vacuum drying may contain the active ingredient along with bulking agents and pH adjusting agents or buffer systems.

Temozolomide formulations for vacuum drying may contain bulking agents like mannitol, lactose, sucrose, sodium chloride, trehalose, dextrose, starch, hydroxyethylstarch (hetastarch), cellulose, cyclodextrins, glycine, or mixtures thereof.

In a preferred embodiment, the bulking agent in the pharmaceutical formulation is mannitol or sodium chloride or sorbitol or sucrose or dextrose or lactose. When a bulking agent is used in the pharmaceutical formulation, the proportion can range from about 5 wt % to about 90 wt %, preferably from about 15 wt % to about 85 wt %.

The formulations for vacuum drying may further comprise pH adjusting agents or buffer systems.

Suitable buffers which can be included in the pharmaceutical formulation include citrate buffers, acetate buffers, phosphate buffers, Acetic acid, Lactic acid, amino acids, tris-buffer, meglumine and the like, and pH adjusting agents that are acidic or alkaline in nature.

The formulation may additionally involve a solvent system. This system can be water or a mixture of water and an organic solvent like ethanol or tertiary-butyl alcohol or isopropyl alcohol or methanol or methylene chloride or ethyl acetate or acetone.

In the vacuum drying process, vacuum promotes liquid evaporation at much lower temperatures than a conventional atmospheric hot air dryer.

As evaporation occurs vapor pressure pushes the vapors into the integrally top-mounted vacuum stack. Here, the large diameter of the stack is sufficient to prevent the vapors from reaching transport velocity and carrying product out of the dryer. The vapor then enters a condenser where exposure to low temperatures causes it to condense back into a liquid. The drop in vapor pressure across the condenser creates a vapor pressure differential within the system which pulls vapor from the dryer to the condenser. The condensate then flows into a recovery or holding tank, especially advantageous when expensive solvents are being used. The entire system vacuum is maintained by a vacuum pump capable of maintaining a medium vacuum level.

The resultant product can be filled in primary packaging containers using any of the dry powder filling techniques known in the art.

### Dry Mix/Dry Powder Compositions

Temozolomide preparation of the present invention can also be made by dry powder filling after mixing the active component with the appropriate excipients. These excipients are designed to add bulk or/and promote the stability of the composition.

These compositions may contain bulking agents like mannitol, sodium chloride and the like. The compositions may also contain stabilisers and pH adjusting agents like tartaric acid.

### Ready to Use Solutions

One of the objects of the invention is to provide ready to use parenteral solutions of Temozolomide. These formulations do not require reconstitution before intravenous administration.

Temozolomide preparation of the present invention can also be made as ready to use solutions after mixing the active component with cosolvents & stabilizers. The cosolvents used for dissolving Temozolomide are Dimethyl sulfoxide, Dimethyl acetamide, N-Methyl Pyrrolidone, and mixtures thereof. Additionally the ready to use solutions may contain buffers, stabilizers, tonicity adjusting aids, ethanol, propyleneglycol, glycofurol, diethyleneglycol monoethyl ether, Polyethyleneglycol and or pH adjusting aids. The ready to use solutions can be filtered and filled into primary packs like glass vials or polymer vials or prefilled syringes.

### WORKING EXAMPLES

### I. Lyophilised Compositions

### Example-1

| S. No | Ingredients | Quantity/vial (mg) | |
|---|---|---|---|
| 1 | Temozolomide | 100 | |
| 2 | Mannitol | 600 | |
| 3 | Tartaric acid pure | 37 | |
| 4 | Water for injection | q.s to 25 | ml |

### Procedure

WFI was cooled to room temperature. Mannitol and buffer were added and dissolved. pH of the solution was adjusted to about 4.5 using 0.1 N HCl/NaOH. Temozolomide was added and stirred for 30 minutes till it completely dissolves. The complete procedure was carried out under nitrogen atmosphere.

The characteristics of the bulk solution before lyophilisation are as follows:

Description: Clear solution
pH: 4.53
Osmolarity: 169 mOsm/kg

The solution was filled into glass vials and lyophilised as per the procedure described previously.

### Example-2

| S. No | Ingredients | Quantity/vial (mg) | |
|---|---|---|---|
| 1 | Temozolomide | 100 | |
| 2 | Mannitol | 600 | |
| 3 | Sodium acetate | 80 | |
| | pure | | |
| 4 | Water for injection | q.s to 25 | ml |

Same procedure for manufacturing was adopted as described in example-1. The characteristics of the bulk solution before lyophilisation are as follows:

Description: Clear solution
pH: 4.55
Osmolarity: 270 mOsm/Kg

The formulations were analysed and the data is tabulated below:

### Physical Parameters:

| Parameter | Example-1 | Example-2 |
|---|---|---|
| Appearance | White to off white cake | White to off white cake |
| Whether the cake is | Intact Freeze dried cake | Intact Freeze dried cake |
| intact on storage | without collapse | without collapse |

### Chemical Parameters:

| Parameter | Drug substance | Example-1 | Example-2 |
|---|---|---|---|
| % Assay | 100.3 | 100.2 | 99.7 |
| % Impurities | | | |
| AIC | 0.02 | 0.07 | 0.4 |
| MTC-1 | Not detected | Not detected | 0.06 |
| Any other maximum | Not detected | Not detected | Not detected |
| impurity | | | |
| Total impurities | 0.04 | 0.08 | 0.52 |

The above formulations were also analysed for the XRD:

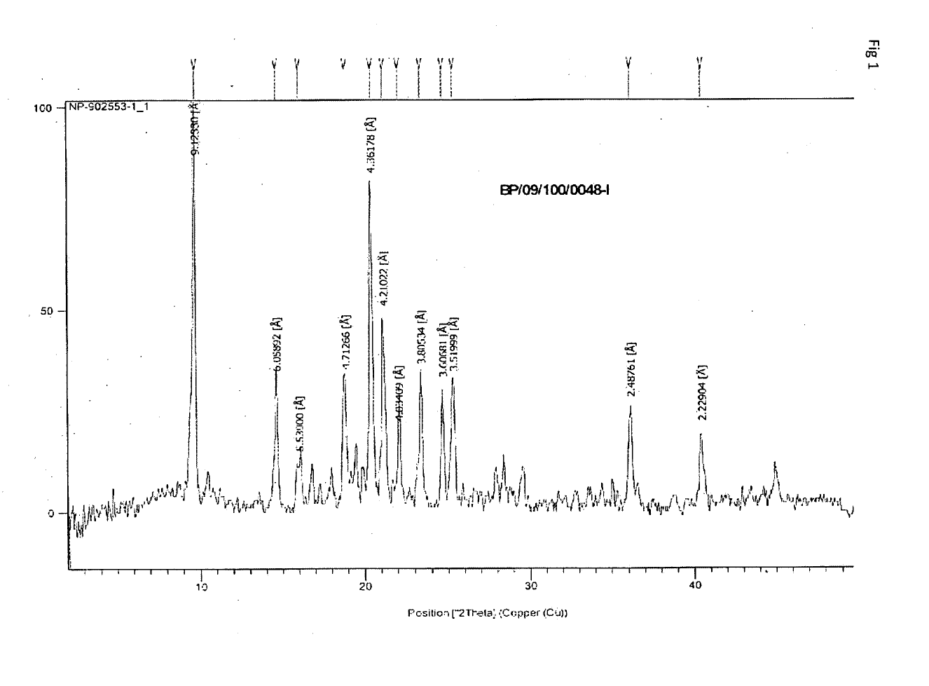

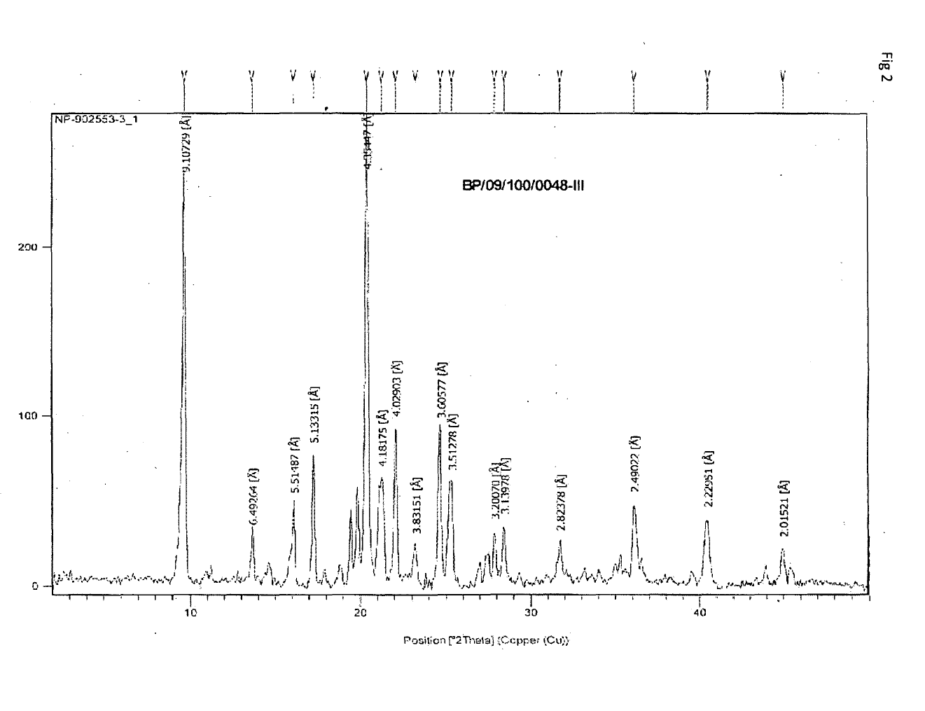

FIG. 1: XRD data generated on the formulation of Example-1
FIG. 2: XRD data generated on the formulation of Example-2

### II. Vacuum Dried Compositions

### Example-1

| S. No | Ingredients | Quantity/vial (mg) | |
|---|---|---|---|
| 1 | Temozolomide | 100 | |
| 2 | Mannitol | 600 | |
| 3 | Tartaric acid pure | 40 | |
| 4 | Water for injection | q.s to 25 | ml |
| 5 | Ethanol | 10 | ml |

Mannitol, and Tartaric acid were dissolved in water at room temperature. Subsequently drug is dissolved in the solution and Ethanol was added. The solution was filtered and vacuum dried. Optionally the solution is dried using spray drier.

### III. Dry Mix/Dry Powder Compositions

### Example-1

| S. No | Ingredients | Quantity/vial (mg) |
|---|---|---|
| 1 | Temozolomide | 100 mg |
| 2 | Mannitol | 100 mg |
| 3 | Pharmaceutically | 0 mg to 60 mg |
| | acceptable buffer (to | |
| | provide pH in range | |
| | of 2.0 to 6.0) | |

### Example-2

| S. No | Ingredients | Quantity/vial (mg) |
|---|---|---|
| 1 | Temozolomide | 100 mg |
| 2 | Sodium chloride | 18 mg |
| 3 | Pharmaceutically | 0 mg to 60 mg |
| | acceptable buffer (to | |
| | provide pH in range | |
| | of 2.0 to 6.0) | |

### Example-3

| S. No | Ingredients | Quantity/vial (mg) |
|---|---|---|
| 1 | Temozolomide | 100 mg |
| 2 | Sorbitol or Sucrose | 100 mg |
| | or Lactose | |
| 3 | Pharmaceutically | 0 mg to 60 mg |
| | acceptable buffer (to | |
| | provide pH in range | |
| | of 2.0 to 6.0) | |

### Procedure:

Temozolomide and excipients are mixed and filled in primary packaging containers. When used for parenteral use the drug and excipients are filled in primary packaging containers using aseptic technique.

### IV. Ready to Use Solutions

### Example-1

| S. No | Ingredients | Quantity/vial (mg) | |
|---|---|---|---|
| 1 | Temozolomide | 100 | |
| 2 | Buffer | 2 | mg |
| 3 | Ethanol | 0 to 1 | mL |
| 4 | DMSO | 3.5 | mL |

### Procedure

DMSO, Ethanol and buffer were mixed. Temozolomide was added to the solution and dissolved. The solution is processed using aseptic technique and filled in sterile containers for storage and subsequent use.

### Example-2

| S. No | Ingredients | Quantity/vial (mg) | |
|---|---|---|---|
| 1 | Temozolomide | 100 | |
| 2 | Buffer | 2 | mg |
| 3 | Ethanol | 0 to 1 | mL |
| 4 | Dimethyl acetamide | 3.5 | mL |

### Procedure

DMA, Ethanol and buffer were mixed. Temozolomide was added to the solution and dissolved. The solution is processed using aseptic technique and filled in sterile containers for storage and subsequent use.

What is claimed is:

1. A parenteral formulation of Temozolomide comprising an excipient selected from the group consisting of bulking agents, buffers, pH adjusting agents, with the proviso that the formulation is free of dissolution enhancing agents.

2. The formulation of claim 1 wherein the composition is lyophilised.

3. The formulation as claimed in claim 1, wherein the bulking agent is selected from the group consisting of mannitol, lactose, sucrose, sodium chloride, sorbitol, trehalose, dextrose, lactose, starch, hydroxyethylstarch (hetastarch), cellulose, cyclodextrins, glycine or mixtures thereof.

4. The formulation of claim 3, wherein the bulking agent is in the range of 5_wt % to 90_wt %.

5. The formulation as claimed in claim 1, wherein more preferably, the bulking agent is in the range of 15_wt % to 85_wt %.

6. The formulation of claim 1, wherein the buffer is selected from the group consisting of citrate buffers, acetate buffers, phosphate buffers, acetic acid, lactic acid, amino acids, tris-buffer and meglumine.

7. The formulation as claimed in claim 1, wherein the pH adjusting agents are acidic or alkaline in nature.

8. The formulation as claimed in claim 1, wherein the organic solvent is ethanol or tertiary-butyl alcohol.

9. The formulation as claimed in claim 1, wherein the formulation is suitable for intravenous administration in the dose ranging from 5 mg to 1000 mg/vial.

10. The formulation as claimed in claim 9, wherein more preferably the formulation suitable for intravenous administration is in the dose ranging from 80 mg to 800_mg/vial and more preferably 100_mg/vial.

11. A process for making the parenteral formulation of Temozolomide injection that is free of dissolution enhancing agents as claimed in claim 1, the process comprising:
(a) dissolving excipients and buffer in water for injection;
(b) dissolving the Temozolomide in the solution formed in the step of dissolving in a concentration of between 0.1%_w/v to 5%_w/v;
(c) lowering the temperature of the Temozolomide preparation to below at least −15° C.;
(d) increasing the temperature of the Temozolomide preparation from <0° C. to about 40° C.; and
(e) subliming the water from the Temozolomide preparation, wherein the formulation has a moisture content of not more than 6.0% by weight.

12. The formulation of claim 1, wherein the formulation is made by a vacuum drying process.

13. The formulation of claim 1, wherein the formulation is made by dry mixing and filling process.
